(19) United States
(12) Patent Application Publication
(10) Pub. No.: US 2022/0096013 A1
(43) Pub. Date: Mar. 31, 2022

(54) BIOINFORMATION MEASURING DEVICE

(71) Applicant: Cardio Ring Technologies, Inc., San Jose, CA (US)

(72) Inventors: Kuang-Fu CHANG, Taichung City (TW); Yu-Chi WANG, Taipei (TW); Wen-Pin SHIH, Taipei (TW)

(73) Assignee: Cardio Ring Technologies, Inc., San Jose, CA (US)

(21) Appl. No.: 17/543,397

(22) Filed: Dec. 6, 2021

Related U.S. Application Data
(63) Continuation of application No. PCT/US2020/044085, filed on Jul. 29, 2020.
(60) Provisional application No. 62/879,536, filed on Jul. 29, 2019.

(51) Int. Cl. A61B 5/00; A61B 5/022

(52) U.S. Cl. CPC A61B 5/6838; A61B 5/6826; A61B 2562/0238; A61B 5/0059; A61B 2562/164; A61B 5/02241

(57) ABSTRACT

The present invention provides a bioinformation measuring device having a first device body, a second device body, and a transducer assembly. The first device body has a first inner surface, and the second device body has a second inner surface facing the first inner surface. An accommodation space for finger is surrounded by the first inner surface and the second inner surface. The transducer assembly is disposed at the second inner surface and includes a finger contact surface facing the first inner surface. The finger contact surface is movable in a first direction perpendicular to the finger contact surface.

### CROSS-REFERENCE TO RELATED APPLICATIONS

This application is a continuation of International Patent Application No. PCT/US2020/044085, filed on Jul. 29, 2020, which claims the benefit of U.S. Provisional Application No. 62/879,536 filed on Jul. 29, 2019, the contents of which are incorporated herein by reference in their entities.

### BACKGROUND OF THE INVENTION

Various non-invasive vital sign monitoring devices have been commercialized, driven by the technology development of medical devices and growing attention in health care of contemporary society. The vital signs that represent important health indices of human beings include glucose concentration, blood pressure, pulse, heart rate, and oxygen saturation etc. To make a vital sign monitoring device portable and to allow users conducting health check on demand, existing devices have been made in the form of a finger clip.

Typically, a finger-clip vital sign monitoring device has a sensor in contact with user's finger pulp. Such sensor can be composed of infrared emitter and receiver. It can be used to calculate oxygen saturation based on different absorption coefficients of different compositions in blood. It can also be used to calculate pulse rate by utilizing photoplethysmography. Another finger clip device, revealed by Hon (U.S. Pat. No. 5,511,546), has a pressure sensor in contact with user's finger pulp to measure deformation and deformation rate of vessels at diastole and systole. Accordingly, pulse or blood pressure can be calculated.

When a conventional finger clip device is applied to a thicker finger, the upper clip cannot completely close, resulting in incomplete contact on the finger under test. Therefore, the finger cannot be stably fixed in the device, causing the increase of sensing noise and the degradation of sensing accuracy. Furthermore, the incomplete closure of the upper clip causes larger deformation of the spring that governs the pivotal rotation of the clip device. As a result, there will be larger restoration force exerting by the spring on the finger to cause discomfort.

In order to make stable and firm contact between a finger pulp and sensor on a finger clip device, current finger-clip vital sign monitoring device suffers from the drawback of causing user's discomfort. Furthermore, it is difficult to visually inspect the alignment and the contact properness of the finger pulp to the sensor due to current clip mechanism design. The misalignment of finger pulp to the sensor often occurs, resulting in weak sensing signals or inaccurate vital sign measurement. In these regards, current vital sign monitoring devices are not satisfactory and has room for improvement.

### BRIEF SUMMARY OF THE INVENTION

The present disclosure includes methods and devices for improved measuring of bioinformation from a finger. The bioinformation can be any information that measures biological information from a finger of an individual. Such bioinformation includes, but is not limited to, blood pressure, pulse, glucose level, oxygen saturation, temperature, etc.

Many of the variations of the present disclosure include measurement devices that are configured to house a finger of the individual to obtain measurement of the bioinformation. However, additional variations can employ one or more sensor assemblies as discussed herein, where the sensor assemblies can be incorporated into a structure that is suited for measurement of bioinformation from another body part.

A first variation of a bioinformation measuring device for monitoring bioinformation from a blood vessel in a finger includes a first device body having a first inner surface; a second device body having a second inner surface, where the first inner surface faces the second inner surface to define an accommodation space configured for positioning of the finger; and a sensor assembly comprising a contact surface and a sensor element, the sensor assembly located in the second device body and having a contact member with a contact surface that extends into the accommodation space, wherein the contact member is coupled to the lower body by a connection region which elastically deforms to a greater degree than the contact member to permit the contact surface to move in a first direction into the second device body and out of the accommodation space when engaged by the finger.

A variation of the device can include a contact member where a bottom surface of the contact member is coupled to the connection region such that a remainder of the contact region extends into the accommodation space wherein the sensor further comprising a contact member, where the contact surface comprises an end of the contact member. In additional variations the contact member (or a portion thereof) is elastic and can deform along the first direction. However, variations of the device include a contact member that is stiffer than the connection region.

Variations of the device can include a second inner surface that comprises an opening, and where the connection region is connected to the second inner surface and surrounds the contact member to suspend the contact member in the opening.

In additional variations of the device, one or both of the device bodies includes an observation section that allows visualization of the accommodation space and the contact surface through the first device body. This feature allows a user to align a finger with the sensor assembly. The observation section can comprise a transparent or translucent material. In an additional variation, the observation section comprises an opening sufficient to visualize the sensor assembly but sufficiently small so as to not interfere with positioning of a finger in the accommodation space.

Variations of the device can further include one or more spring elements coupled to the first device body and the second device body at an end of the first device body and second device body that is opposite to the accommodation space, wherein a pin extends through a first slot in the first device body and a second slot in the second device body such that the spring element biases the first device body to an open position from a closed position.

In some cases, the pin can be undersized relative to the first slot or the second slot such that the first device body and second device body can displace in an opposite direction when biased in the closed position.

Additional variations of the devices can further comprise a first limit surface coupled to the first device body, where the first limit surface engages the second device body in the closed position to prevent further closure of the first device body relative to the second device body in the closed position and a second limit surface coupled to the first device body, where the second limit surface engages the second device body in the open position to prevent further opening of the first device body relative to the second device body in the open position. In one example, the first limit surface and second limit surface are located on a limit rim disposed between the first device body and second device body.

Any of the variations of the devices described herein can include an electromechanical sensor assembly, an optic sensor assembly, or any sensor assembly that permits measurement of the bioinformation.

For example, where the sensor element can comprise an electro-mechanical transducer and where the contact member is moveably coupled to the transducer such that when the finger is positioned against the contact surface movement of the contact member caused by pressure changes in the blood vessel cause the contact member move against the transducer to produce a signal for determining bioinformation.

In another variation, sensor assembly further comprises an illumination source configured to transmit illumination to the accommodation space and the sensor element comprises a light sensor, where the contact member is configured to transmit illumination reflected by the finger in the accommodation space to the light sensor to produce a signal for determining bioinformation.

Another variation of the device can include a bioinformation measuring device having a first device body, a second device body, and a transducer assembly. The first device body has a first inner surface, and the second device has a second inner surface facing the first inner surface. An accommodation space for finger is surrounded by the first inner surface and the second inner surface. The transducer assembly is disposed at the second inner surface and includes a finger contact surface facing the first inner surface. The finger contact surface is movable in a first direction perpendicular to the finger contact surface.

In one variation of the device including a first device body, a second device body, and a transducer assembly, the first device body has a first inner surface, and the second device body has a second inner surface facing the first inner surface. An accommodation space for finer is surrounded by the first inner surface and the second inner surface. The transducer assembly is disposed at the second inner surface and includes a finger contact surface facing the first inner surface. The first device body further includes a transparent assembly. The projection area of the transparent assembly on the second inner surface overlaps at least partially the finger contact surface of the transducer assembly. The transparent assembly penetrates through the first device body so that at least a light can propagate through a first outer surface and the first inner surface of the first device body.

Comparing to conventional finger clip devices, the bioinformation measuring device in the present invention can maintain sufficient contact area between the first device body and the finger when applied to a thicker finger. The present device allows the finger to be stably fixed inside an accommodation space to reduce sensing noise, to increase measurement accuracy, and to improve comfort of the finger.

### BRIEF DESCRIPTION OF THE SEVERAL VIEWS OF THE DRAWINGS

FIG. 1A illustrates a sideview of a first embodiment of a bioinformation measuring device of the present invention.

FIG. 1B illustrates an application of the bioinformation measuring device of FIG. 1A.

FIG. 2 illustrates a perspective view of the device of FIG. 1A but in an open configuration.

FIGS. 3A and 3B represent cross sectional views of the bioinformation measuring of FIG. 1 taken along the line A-A for an electromechanical transducer assembly an optical transducer assembly respectively.

FIGS. 4A and B illustrates the finger contact surface of the transducer assembly subjected to an external force for the respective assemblies of FIGS. 3A and 3B.

FIG. 5 illustrates a variation of FIG. 3A or 3B where the contact member has a covering.

FIG. 6 illustrates a variation of the bioinformation measuring device of FIG. 1.

FIG. 7 illustrates an explosion drawing of the bioinformation measuring device of FIG. 1.

FIG. 8A illustrates an application of the bioinformation measuring device of FIG. 6.

FIG. 8B illustrates another application of the bioinformation measuring device of FIG. 6.

FIG. 9 illustrates another variation of the bioinformation measuring device of FIG. 1.

FIG. 10A illustrates an application of the bioinformation measuring device of FIG. 9.

FIG. 10B illustrates another application of the bioinformation measuring device of FIG. 9.

FIG. 11 illustrates the three-dimensional view of a second embodiment of a bioinformation measuring device of the present invention.

FIG. 12 illustrates an application of the bioinformation measuring device of FIG. 11. 120

FIG. 13 illustrates a variation of the bioinformation measuring device of FIG. 11.

### DETAILED DESCRIPTION OF THE INVENTION

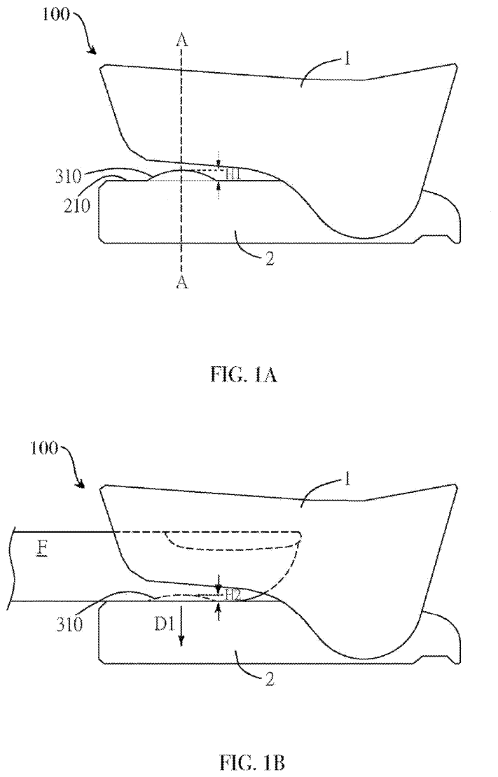

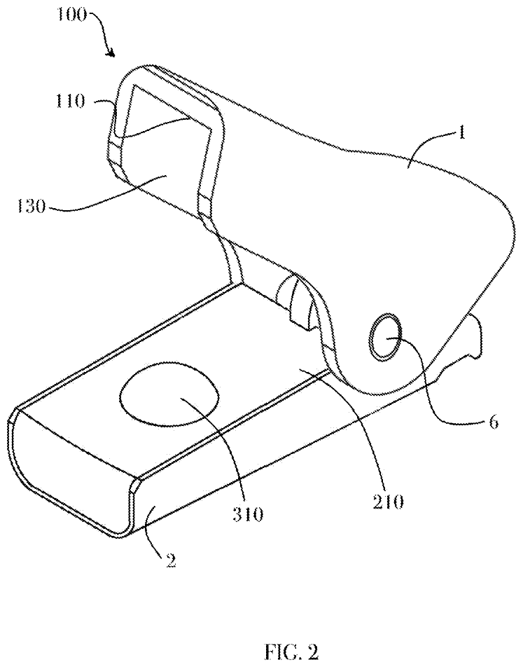

FIG. 1A illustrates sideview of a first variation of a bioinformation measuring device 100 that comprises a first device body 1, a second device body 2, and a sensor assembly having a contact surface 3. The first device body 1 has a first inner surface, and the second device body 2 has a second inner surface 210 facing the first inner surface. FIG. 2 shows the device 100 in an open position to better illustrate the accommodation space 130 that accepts a finger F for testing in the assembly 100. As shown in FIG. 1B, where the finger F enters the accommodation space 130 and engages the first surface 110 and the second inner surface 210. The contact surface of the sensor assembly 3 is disposed at the second inner surface 210 of the second device body 2 and has a finger contact surface 310 facing the first inner surface 110. Specifically, the accommodation space 130 is to accommodate a finger under test, and the finger contact surface 310 of the transducer assembly 3 is to contact the measurement surface of the finger in order to receive bioinformation. Typically, the measurement surface is, but not limited to, on finger pulp of the finger. The finger contact surface 310 is movable in a first direction D1 that is generally perpendicular or away from to the first inner surface.

FIG. 1B illustrates an application of the bioinformation measuring device of FIG. 1. When the finger F is inserted in the accommodation space defined by the space between the upper device body and the lower device body and the finger F engages the contact surface 310 of the sensor assembly 3, the contact surface 310 moves (generally in direction D1) due to the force exerted by the finger. As a result, the height of the finger contact surface 310 relative to the second inner surface 210 decreases from height H1 of FIG. 1A to height H2 of FIG. 1B. The ability of the contact surface 310 of the sensor assembly to move back and forth along direction D1 allows the transducer assembly (discussed below) to receive more accurate bioinformation from vessel in the finger F at a relative shorter distance

FIG. 2 illustrates an example of a device 100 where the upper device body 1 is in an open position relative to a lower device body 2. FIGS. 1A and 1B illustrate the device 100 in a closed configuration. As shown in FIG. 2, the contact surface 310, which is part of or adjacent to a contact member (not shown) extends beyond the second inner surface 210 so that when in the closed position the contact surface 310 and contact member extend into the accommodation space (which is the region between surfaces 110 and 210). As shown the upper device body 1 can be rotatably coupled to the lower device body 2 by any conventional means, including the use of a pin or shaft 6 about which the upper device body 1 rotates to move from an open configuration to a closed configuration.

The devices 100 described herein can include any number of sensor assemblies. For example, when the sensor assembly is an electro-mechanical assembly, the sensor assembly includes a pressure sensor (such as a piezo electric transducer), and the bioinformation includes blood pressure information or pulse information. However, the bioinformation in this present invention is not limited to blood pressure information and pulse information. In other embodiments of the present invention, the transducer assembly 3 could include pressure sensor and optical sensor to simultaneously receive information related to blood pressure, pulse, glucose concentration, heart rate, and oxygen saturation etc.

Conventional devices can cause discomfort to a finger confining the finger with an upper cap and a lower cap, and meanwhile, protruding into the finger pulp with a sensor. In the devices disclosed herein, finger is placed under less pressure the pressure exerting on the finger is reduced due since the contact member/contact surface is movable along the first direction D1s.

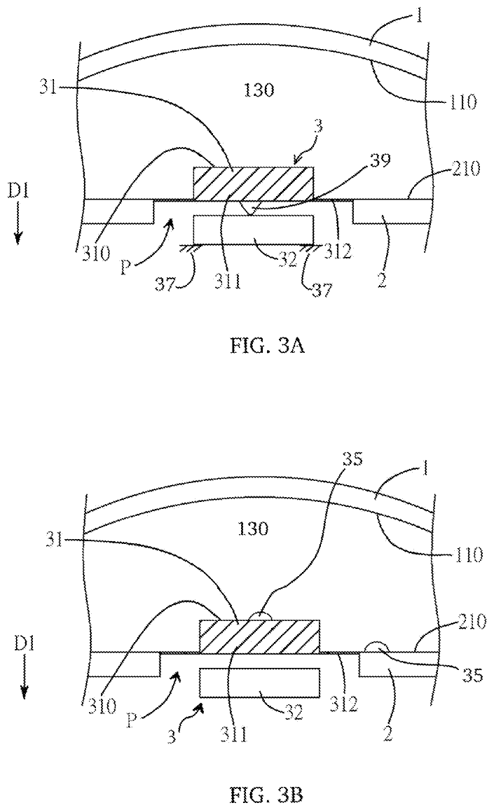

FIGS. 3A and 3B represent cross sectional views of the bioinformation measuring of FIG. 1 taken along the line A-A from FIG. 1A for an electromechanical sensor assembly 3 (FIG. 3A) and an optical sensor (FIG. 3B). For purposes of demonstrating the concepts of the disclosure, the sensors are not illustrated with electrodes or other circuitry that would be readily apparent to those skilled in the art. As shown, variations of a sensor assembly 3 can include a contact member 31 and at least a sensor 32. In the case of an electromechanical sensor (such as a piezoelectric assembly) the sensor assembly 32 will be secured (represented by anchors 37) within the device and adjacent a driver or other mechanical member 39 that transmit motion of the contact member 31 to the sensor 32. In the variation shown in FIG. 3B, an optical assembly 3 can include any number of illumination sources 35 optionally positioned on the contact member 31 and/or the second body device 2. The illumination sources 35 can be LED, optical fibers, or other sources of illumination that produce reflected energy from the finger that is transmitted through the contact member 31 and to the sensor 32. In the variation shown in FIG. 3B, the sensor 32 can either be fixed to the contact member 31 (and free-floating relative to the second body device 2) or can be fixed to the second body device 2).

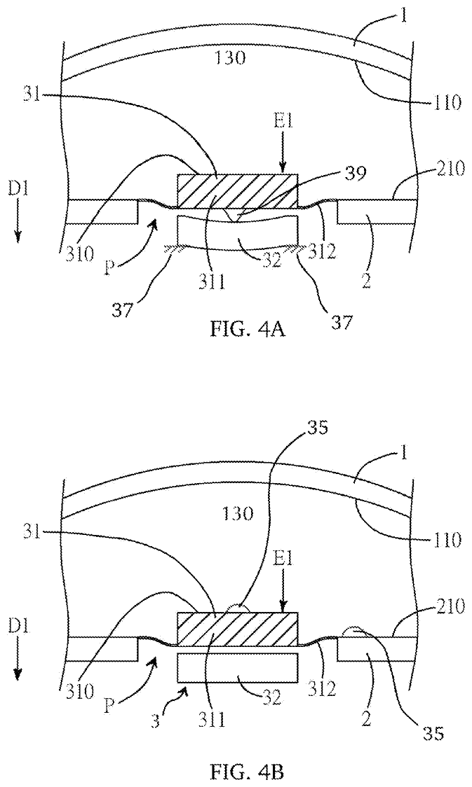

The contact member 31 is positioned between the sensor 32 and the accommodation space 130. As discussed above, the contact member 31 is moveable in direction D1 to allow the sensor assembly 3 to be movable back and forth along direction D1. The contact member 31 is connected to the second device body 2, by a connecting region that deflects to allow movement of the contact member 31. FIGS. 4A and 4B depict the contact surface 310 of the sensor assembly of FIGS. 3A and 3B subjected to an external force E1. As shown in FIG. 4A displacement of the contact member 31 causes the driver or mechanical member 39 to move towards the sensor 32 causing displacement of the sensor 32 as shown. The assembly 3 transmits this displacement into a signal that can be transformed to determine bioinformation. In practice, movement of the contact member 31 against the sensor 32 does not allow differentiation between forces from movement of a finger and forces caused by pulsatile flow of blood in the finger. However, such movements can be differentiated using signal processing. For example, pulsatile flow is usually periodic, but finger motion is random. Moreover, the initial displacement caused by the finger pushing against the contact member 31 can cause a significant displacement relative to the small displacements when pulsatile blood flow in the finger causes movement of the contact member 31 to cause deformation of the sensor. In contrast, in the optical assembly of FIG. 4B, the sensor 32 can be mechanically coupled to the contact member 31 or can be fixed independently of the contact member 31 as long as the sensor 32 does not impede motion of the contact member 31. In alternate variations of optical sensor assemblies, the sensor 32 can be used to limit movement of the contact member 31.

The second inner surface 210 can have an opening P in which the contact member 31 is suspended. The sensor 32 can also be disposed in the opening P at a farther distance from the accommodation space 130 than to the second inner surface 210. The contact member 31 can also form a central region 311 of the sensor assembly 3 where the connection region 312 connects the contact member 31 to an edge of the opening P. In addition, the connection region 312 can be elastically deformable in the first direction D1. In variations of the device, a stiffness of the central region 311 is greater than that of the connection region 312 due to the presence of the contact member 31 so that the external force E1 exerting on the central region 311 can be partially transmitted to the connection region 312. Accordingly, when an external force E1 in the accommodation space 130 is exerted along the first direction D1 on to the central region 311, part of the external force E1 will be transmitted from the central region 311 to the connection region 312, causing the connection region 312 to deform along the first direction D1. As a result, the central region 311 will move in toward the opening R and thus transmit the pressure information of the external force E1 to the sensor 32.

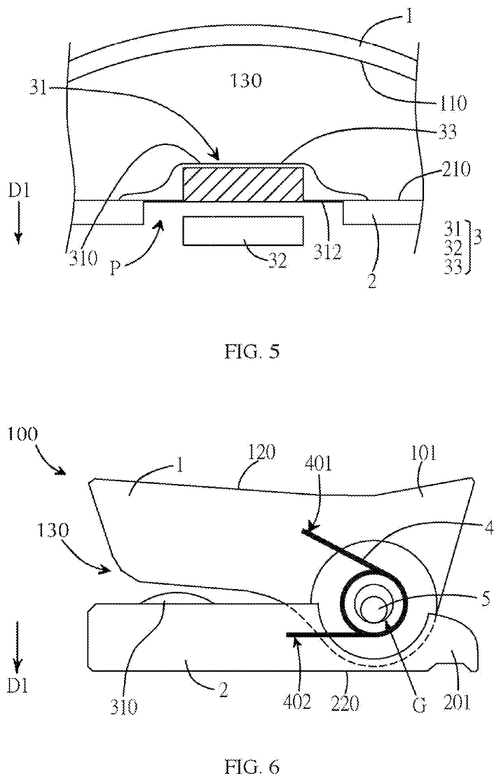

The present invention is not limited to the variation that is illustrated by FIGS. 3 and 4. Additional variations of the present device include the sensors 32 that are disposed inside the elastic contact member 31 and can move together with the elastic contact member 31 along the first direction D1 in toward the opening P. FIG. 5 illustrates a variation having a sensor assembly 3 with an elastic membrane 33 that forms a tissue contacting surface 310 and covers the contact member 31 and interfaces the accommodation space 130 in order to improve comfort of finger in touch with the finger contact surface 310.

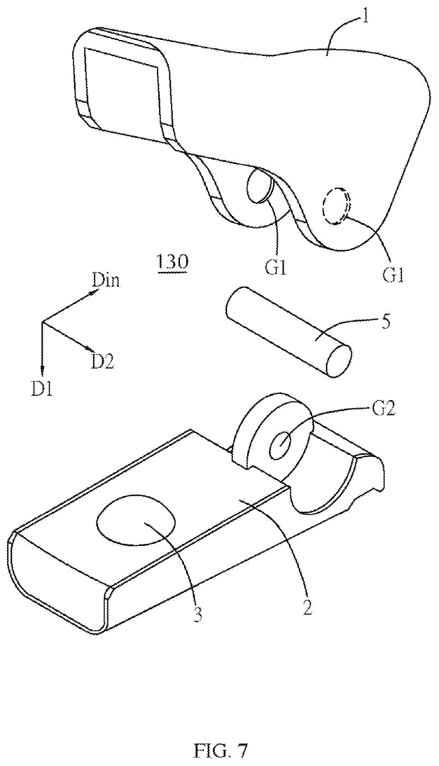

FIG. 6 illustrates a variation of the bioinformation measuring device 100 of the present invention. In this variation, the first device body 1 and the second device body 2 of the bioinformation measuring device 100 are joined with a spring 4 and a shaft or pin 5 that functions as a locating element. Therefore, the first device body 1 and the second device body 2 can be open or close about a finger through relative rotation to the center of the spring 4. Specifically, the spring 4 has a relatively extendable first spring end 401 and a second spring end 402. The first spring end 401 connects to the first device body 1, and the second spring end 402 connects to the second device body 2. Part of the first device body 1 and part of the second device body 2 jointly form the limit slot G. The locating element 5 is disposed in the limit slot G. Because the limit slot G is jointly formed by part of the first device body 1 and part of the device body 2, the locating element 5 has the effect of limiting the relative position between the first device body 1 and the second device body 2.

Furthermore, a limit slot G of the present invention can be illustrated by the explosion drawing of FIG. 7. For the convenience of showing the structures of the limit slot G, FIG. 7 displays only the first device body 1, the second device body 2, and the limit slot G. The accommodation space 130 has a finger insertion direction Din and a second direction that is orthogonal to the first direction D1 and the finger insertion direction Din. The first device body has two side slots G1 facing each other in the second direction D2. The second device body 2 has a through hole G2 in the second direction D2. When the first device body 1 and the second device body 2 are assembled by aligning the side slots G1 to the through hole G2, the side slots G1 and the through hole G2 together form the limit slot G. The locating element 5 extends inside the side slots G1 and the through hole G2 in the second direction 2 so that the relative position between the first device body 1 and the second device body 2 is confined. The assembly of the limit slot G of the present invention is not limited to the illustration of FIG. 7. In other variations, the through hole G2 can be in the first device body 1, and the side slots G1 are on the second device body 2. In this case, the locating element 5 extends inside the through hole G2 of the first device body 1 and the side slots G1 of the second device body 2.

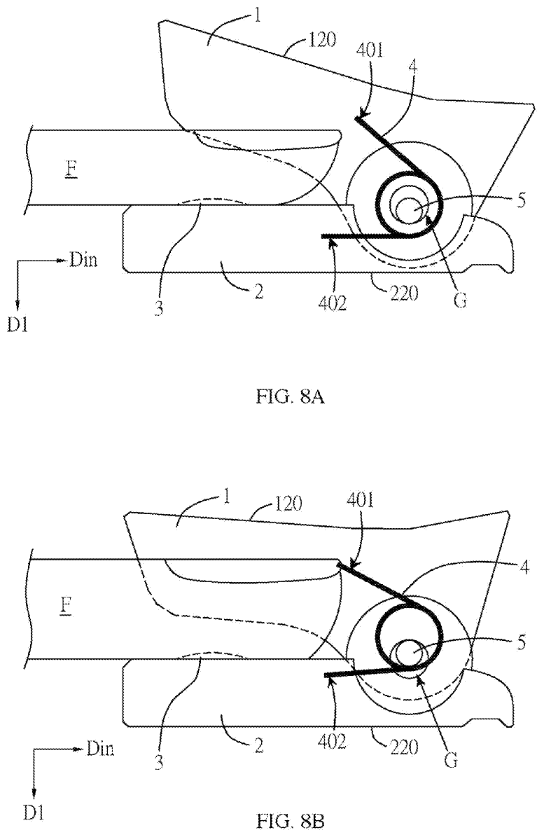

FIG. 6 illustrates a variation of the bioinformation measuring device of FIG. 1, and FIG. 8A illustrates an application of such bioinformation measuring device. To enlarge the accommodation space 130 of the bioinformation measuring device 100, under the setup of the spring 4 and the locating element 5, an user can apply a force on the outside of the device 100. For example, applying a force along the first direction D1 on the first outer surface 120 at the distal end of the first device body 101 and another force in the opposite of the first direction D1 on the second outer surface 220 at the distal end of the second device body 201. When the accommodation space 130 is enlarged, a finger F can be inserted along direction Din into the accommodation space 130, as illustrated in FIG. 8A. When the finger F is positioned on top of the transducer assembly 3, the user can remove the applied forces. As a result, due to the restoration of the spring 4, the first spring end 401 and the second spring end 402 of the spring 4 approaches each other to close the bioinformation measuring device 100. Therefore, the finger F is fixed between the first device body 1 and the second device body 2.

Furthermore, the locating element 5 extends through the center of the spring 4, as illustrated in FIG. 6. Therefore, the spring 4 and the locating element 5 can displace together, and the locating element 5 can move back and forth in the first direction D1 inside the limit slot G. FIG. 8B illustrates a finger F, that is thicker in the first direction D1, inside the accommodation space 130. The finger F pushes the first device body 1 toward the opposite of the first direction 1. Because the first spring end 401 of the spring 4 connects to the first device body 1, the center of the spring 4 is moved toward the opposite of the first direction D1. Because the locating element 5 and the spring 4 move together, the locating element 5 inside the limit slot G is carried by the spring 4 toward the opposite of the first direction D1. The movement of the locating element 5 is limited when it reaches the top edge of the limit slot G, as illustrated in FIG. 8B. At this condition, the spring 4 cannot move further toward the opposite of the first direction D1. Therefore, when the limit slot G limits the displacement of the location element 5, it also confines the spring 4 and the first device body 1.

With the above-mentioned features of the present invention, the bioinformation measuring device 100 can accommodate a wider thickness range of finger under test. Compared to conventional finger clip devices, the bioinformation measuring device 100 in the devices under the present invention can maintain sufficient contact area between the first device body 1 and the finger F when applied to a thicker finger F. The present invention allows the finger F to be stably fixed inside the accommodation space 130 to reduce sensing noise, to increase measurement accuracy, and to improve comfort of the finger F.

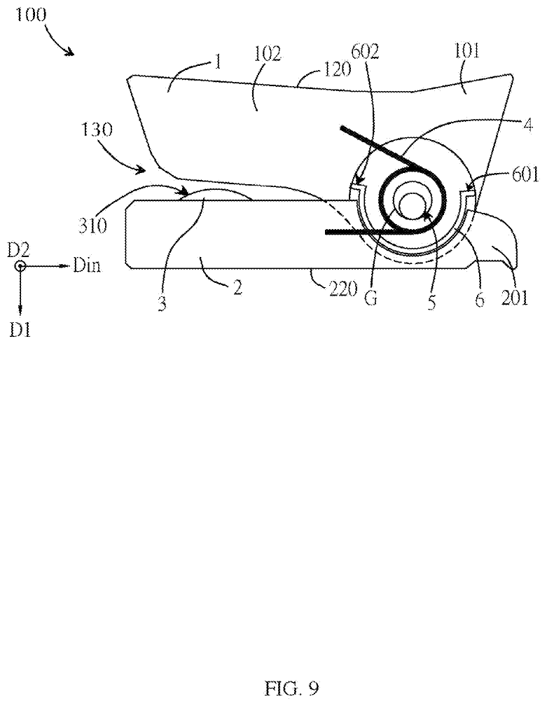

FIG. 9 illustrates another variation of the bioinformation measuring device. Such bioinformation measuring device further comprises a limit rim 6 disposed between the first device body 1 and the second device body 2. The first device body 1 and the second device body 2 are connected by a pivotal assembly to perform relative rotation between a maximum open position and a minimum closure position. An embodiment of the pivotal assembly comprises a spring 4, a locating element 5, and a limit slot G, as illustrated in FIG. 6. However, the present invention is not limited to this embodiment. For example, a variation of the pivotal assembly can be the spring 4 in FIG. 6 with one end toward the distal end of the first device body 101 and the other end toward the distal end of the second device body 201.

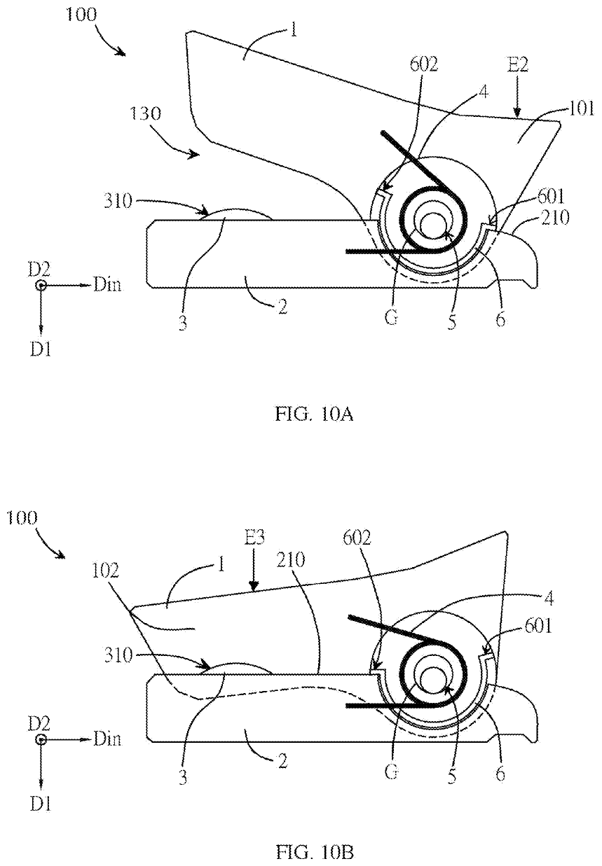

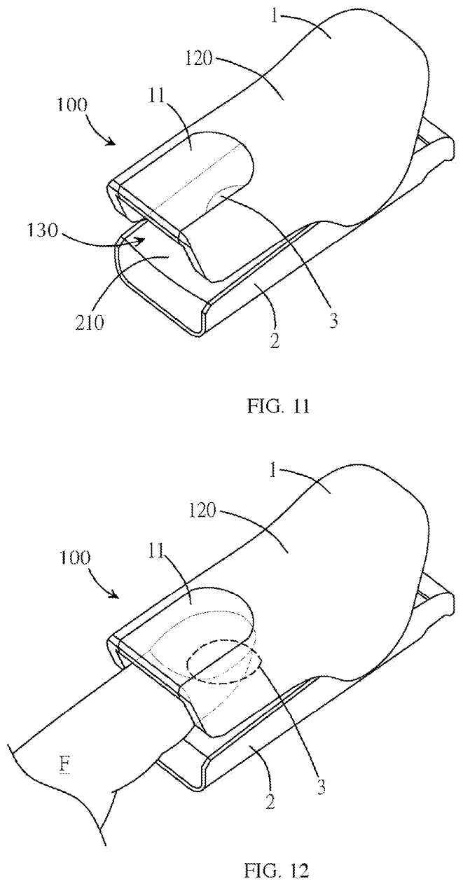

FIG. 9 illustrates another variation of the bioinformation measuring device, and FIG. 10A illustrates an application of such device. FIG. 10A shows the first device body 1 and the second device body 2 in their maximum open position. The limit rim 6 has a first rim end 601 at the side of the pivotal assembly that is opposite to the accommodation space 130. When the distal end of the first device body 101 is subjected to an external force E2, the first device body 1 and the second device body 2 rotates toward the maximum open position. Meanwhile, the first rim end 601 moves toward the second device body 2. When the first device body 1 and the second device body 2 reach their maximum open position, as illustrated in FIG. 10A, the first rim end 601 touches the second inner surface 210 on the second device body 2. Therefore, the limit rim 6 can prevent the spring 4 from being further opened to induce permanent deformation due to large deformation.

FIG. 9 illustrates another variation of the bioinformation measuring device, and FIG. 10B illustrates another application of such device. FIG. 10B shows the first device body 1 and the second device body 2 in their minimum closure position. The limit rim 6 has a second rim end 602 at the side of the pivotal assembly that is close to the accommodation space 130. When the proximal end of the first device body 102 is subjected to an external force E3, the first device body 1 and the second device body 2 rotate toward the minimum closure position. Meanwhile, the second rim end 602 moves toward the second device body 2. When the first device body 1 and the second device body 2 reach their minimum closure position, as illustrated in FIG. 10B, the second rim end 602 touches the second inner surface 210 on the second device body 2. Therefore, the limit rim 6 can prevent the spring 4 from being further compressed. The rim ends 601 and 602 can function as limit surfaces to limit relative movement of the first device body 1 to the second device body.

The rim 6 in the present invention prevents the spring 4 from being overly stretched or compressed so that the restoration capability of the spring 4 and thus the reliability of the bioinformation measuring device 100 can be improved.

With all mentioned above, the first embodiment of a bioinformation measuring device 100 of the present invention can reduce the pressure exerting on the finger by utilizing the feature that the finger contact surface 310 can move back and forth in the first direction D1. Therefore, the comfort to the finger during measurement can be improved.

In addition, the interaction between the spring 4, the locating element 5, and the limit slot G allows the bioinformation measuring device 100 to accept a wider range of finger thickness. Even when the finger is thicker, the first device body can still maintain sufficient contact area with the finger under test. Therefore, the comfort of the finger during measurement can be further improved. Furthermore, the stability of the finger inside the bioinformation measuring device 100 is enhanced so that more accurate measurement can be achieved. The rim 6 that prevents the spring 4 from being overly deformed further improve the reliability of the bioinformation measuring device 100.

A second embodiment of a bioinformation measuring device of the present invention

FIG. 11 illustrates the perspective view of a second embodiment of a bioinformation measuring device of the present invention. In this second embodiment, the components that are identical to those in the first embodiment are labeled with the same notations. FIG. 12 illustrates an application of the bioinformation measuring device of FIG. 11. The main difference of the second embodiment from the first embodiment is that the first device body 1 of the bioinformation measuring device 100 in the second embodiment has a transparent assembly 11. Particularly, the projection area of the transparent assembly 11 on the second inner surface 210 at least partially overlaps the transducer assembly 3. In addition, the transparent assembly 11 penetrates the first device body 1 to allow light to propagate through the first device body 1 between the first inner surface 110 and the first outer surface 120. Specifically, the transparent assembly 11 in this embodiment allows user to observe the finger F from the first outer surface 120 of the first device body 1 when the finger F is put into the accommodation space 130, as illustrated in FIG. 12. This embodiment enables precise alignment of the finger F to the transducer assembly 3 to further improve measurement efficiency and accuracy. The transparent assembly 11 can comprise an optically clear or optically opaque material that allows observation therethrough. Alternatively, the transparent assembly 11 can comprise one or more openings to permit visualization where the opening does not interfere with the ability of the device to house a finger.

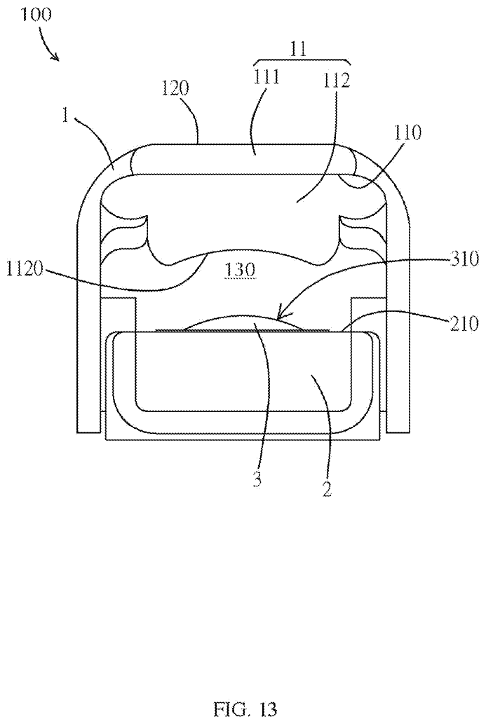

FIG. 13 illustrates a variation of the bioinformation measuring device of FIG. 11. In this variation, the transparent assembly 11 includes a transparent cover 111 that penetrates through the first device body 1 and a transparent soft pad 112 that directly connects to the transparent cover 111 and interface the accommodation space 130. The transparent soft pad 112 can be made of soft materials such as silicone rubber, emulsion, or polyurethane. The transparent soft pad 112 uniformly distributes the clamping pressure of the first device body 1 onto the interface between the transparent soft pad 112 and the finger F. Therefore, the comfort of the finger F is further improved. In this embodiment, the transparent soft pad 112 has a concave surface 1120 interfacing the accommodation space 130 to provide a better fitting with the finger F. As a result, the stability of fixing finger in the bioinformation measuring device 100 is further enhanced. Thus, the sensing noise can be reduced, and the measurement accuracy can be improved.

Although the present invention has been described with reference to the preferred embodiments thereof, it will be understood that the invention is not limited to the details thereof. Various changes and modifications in accordance with the appropriate technical solutions and technical concepts of the present invention should belong to the invention as claimed. Therefore, all such substitutions and modifications are intended to be embraced within the scope of the invention as defined in the appended claims.

Well-known structures, materials, or operations are not shown or described in detail to avoid obscuring aspects of the described devices. As those skilled in the art would realize, the described embodiments may be modified in various different ways, all without departing from the spirit or scope of the present invention. It should be noted that, without conflict, in the embodiment of the present invention and embodiments of features can be combined with each other. Therefore, it should be appreciated that the embodiments described herein are not intended to be exhaustive of all possible embodiments in accordance with the present disclosure, and that additional embodiments may be conceived based on the subject matter disclosed herein.

We claim:

1. A bioinformation measuring device for monitoring bioinformation from a blood vessel in a finger, the device comprising:
a first device body having a first inner surface;
a second device body having a second inner surface, where the first inner surface faces the second inner surface to define an accommodation space configured for positioning of the finger; and
a sensor assembly comprising a contact surface and a sensor element, the sensor assembly located in the second device body and having a contact member with a contact surface that extends into the accommodation space, wherein the contact member is coupled to the lower body by a connection region which elastically deforms to a greater degree than the contact member to permit the contact surface to move in a first direction into the second device body and out of the accommodation space when engaged by the finger.

2. The device of claim 1, where a bottom surface of the contact member is coupled to the connection region such that a remainder of the contact region extends into the accommodation space.

3. The device of claim 2, wherein the contact member is elastic and can deform along the first direction.

4. The device of claim 1, the second inner surface comprises an opening, and where the connection region is connected to the second inner surface and surrounds the contact member to suspend the contact member in the opening.

5. The device of claim 1, wherein the contact member is stiffer than the connection region.

6. The device of claim 1, wherein the first device body comprising an observation section that allows visualization of the accommodation space and the contact surface through the first device body.

7. The device of claim 6, where the observation section comprises a transparent or translucent material.

8. The device of claim 1, further comprising a spring element coupled to the first device body and the second device body at an end of the first device body and second device body that is opposite to the accommodation space, wherein a pin extends through a first the first device body and a second slot in the second device body such that the spring element biases the first device body to an open position from a closed position.

9. The device of claim 8, wherein the pin is undersized relative to the first slot or the second slot such that the first device body and second device body can displace in an opposite direction when biased in the closed position.

10. The device of claim 8, further comprising a first limit surface coupled to the first device body, where the first limit surface engages the second device body in the closed position to prevent further closure of the first device body relative to the second device body in the closed position and a second limit surface coupled to the first device body, where the second limit surface engages the second device body in the open position to prevent further opening of the first device body relative to the second device body in the open position.

11. The device of claim 10, wherein the first limit surface and second limit surface are located on a limit rim disposed between the first device body and second device body.

12. The device of claim 1, where the sensor element comprises an electro-mechanical transducer and where the contact member is moveably coupled to the transducer such that when the finger is positioned against the contact surface movement of the contact member caused by pressure changes in the blood vessel cause the contact member move against the transducer to produce a signal for determining bioinformation.

13. The device of claim 1, where sensor assembly further comprises an illumination source configured to transmit illumination to the accommodation space and the sensor element comprises a light sensor, where the contact member is configured to transmit illumination reflected by the finger in the accommodation space to the light sensor to produce a signal for determining bioinformation.

14. A bioinformation measuring device for monitoring bioinformation in a blood vessel of a finger, the device comprising:
a first device body having a first inner surface;
a second device body having a second inner surface facing the first inner surface so that an accommodation space of finger is surrounded by the first inner surface and the second inner surface; and
a transducer assembly disposed at the second inner surface including a finger contact surface facing the first inner surface, wherein the finger contact surface can move back and forth in a first direction perpendicular to the finger contact surface.

15. The device of claim 14, wherein the transducer assembly further comprising an elastic contact member and a sensor, where the elastic contact member is disposed between the sensor and the accommodation space of finger and can deform in the first direction.

16. The device of claim 15, wherein:
the second inner surface comprising an opening; and
the elastic contact member having a central region that overlaps the center of the opening and at least a connection region that connects the central region to an edge of the opening,
wherein the central region protrudes into the accommodation space relative to the second inner surface, and the sensor is farther from the accommodation space relative to the second inner surface.

17. The device of claim 16, wherein the central region is stiffer than the connection region.

18. The device of claim 14, wherein the first device body comprising a transparent assembly that penetrates through the first device body and has a projection area at least partially overlapping the transducer assembly on the second surface,
wherein the transparent assembly allows at least a light to propagate through the first device body between the first inner surface and a first outer surface of the first device body.

19. The device of claim 14 further comprising a spring and a locating element, wherein
the spring has a first spring end connecting to the first device body and a second spring end connecting to the second device body;
part of the first device body and part of the second device body together form a limit slot;
the locating element is disposed at least partially inside the limit slot and can move back and forth in the first direction inside the limit slot; and
the spring and the locating element move together.

20. The device of claim 19, wherein
the accommodation space of finger has a finger insertion direction and a second direction orthogonal to the first direction and the finger insertion direction;
either the first device body or the second device body has at least a side slot along the second direction; and
either the first device body or the second device body that does not have side slot has at least a through hole along the second direction,
wherein the at least side slot and the at least through hole forms a limit slot and at least part of the locating element extends inside the at least slot and the at least through hole along the second direction.

21. The device of claim 14 further comprising a limit rim disposed between the first device body and second device body, wherein
the first device body and the second device body are connected by a pivotal assembly and can perform relative rotation with respect to the pivotal assembly between a maximum opening position and a minimum closure position; and
the limit rim has a first rim end at the side of the pivotal assembly that is relatively farther from the accommodation space of the finger under test,
wherein the first rim end moves toward the second device body when the first device body and the second device body rotate relatively to the maximum opening position, and the first rim end touches the second inner surface of the second device body when the first device body and second device body reach the maximum opening position.

22. The device of claim 21 wherein the limit rim further comprising a second rim end disposed at the side of the pivotal assembly that is close to the accommodation space of the finger under test, wherein
the second rim end moves toward the second device body when the first device body and the second device body rotate relatively to the minimum closure position, and the second rim end touches the second inner surface of the second device body when the first device body and second device body reach the minimum closure position.

23. The device of claim 14 wherein the sensor is a pressure sensor for the transducer assembly to receive bioinformation from the finger contact surface, and the bioinformation includes blood pressure information.

24. A bioinformation measuring device for monitoring bioinformation in a blood vessel of a finger, the device comprising:
a first device body having a first inner surface;
a second device body having a second inner surface facing the first inner surface so that an accommodation space of finger is surrounded by the first inner surface and the second inner surface; and
a transducer assembly disposed at the second inner surface including a finger contact surface facing the first inner surface, wherein the first device body has a transparent assembly that has projection area at least partially overlaps the transducer assembly on the second inner surface, and the transparent assembly penetrates through the first device body to allow at least a light to propagate through the first device body between the first inner surface and a first outer surface of the first device body.

25. The device of claim 24, wherein the finger contact surface can move back and forth in a first direction that is perpendicular to the finger contact surface.

26. The device of claim 24, wherein the transducer assembly comprises an elastic contact member and a sensor, where the elastic contact member is disposed between the sensor and the accommodation space of finger under test, and the elastic contact member is deformable in the first direction.

27. The device of claim 26, wherein the second inner surface comprising an opening; and
the elastic contact member having a central region that overlaps the center of the opening and at least a connection region that connects the central region to an edge of the opening,
wherein the central region protrudes into the accommodation space relative to the second inner surface, and the sensor is farther from the accommodation space relative to the second inner surface.

28. The device of claim 27, wherein the central region is stiffer than the connection region.

29. The device of claim 24 further comprising a spring and a locating element, wherein the spring has a first spring end connecting to the first device body and a second spring end connecting to the second device body;
part of the first device body and part of the second device body together form a limit slot;
the locating element is disposed at least partially inside the limit slot and can move back and forth in the first direction inside the limit slot; and
the spring and the locating element move together.

30. The device of claim 29, the accommodation space of finger has a finger insertion direction and a second direction orthogonal to the first direction and the finger insertion direction;
either the first device body or the second device body has at least a side slot along the second direction; and
either the first device body or the second device body that does not have side slot has at least a through hole along the second direction,
wherein the at least side slot and the at least through hole forms a limit slot and at least part of the locating element extends inside the at least slot and the at least through hole along the second direction.

31. The device of claim 24 further comprising a limit rim disposed between the first device body and second device body, wherein
the first device body and the second device body are connected by a pivotal assembly and can perform relative rotation with respect to the pivotal assembly between a maximum opening position and a minimum closure position; and
the limit rim has a first rim end at the side of the pivotal assembly that is relatively farther from the accommodation space of the finger under test,
wherein the first rim end moves toward the second device body when the first device body and the second device body rotate relatively to the maximum opening position, and the first rim end touches the second inner surface of the second device body when the first device body and second device body reach the maximum opening position.

32. The device of claim 31 wherein the limit rim further comprising a second rim end disposed at the side of the pivotal assembly that is close to the accommodation space of the finger under test, wherein
the second rim end moves toward the second device body when the first device body and the second device body rotate relatively to the minimum closure position, and the second rim end touches the second inner surface of the second device body when the first device body and second device body reach the minimum closure position.

33. The device of claim 24, wherein the sensor is a pressure sensor for the transducer assembly to receive bioinformation from the finger contact surface, and the bioinformation includes blood pressure information.
